(19) United States
(12) Patent Application Publication
(10) Pub. No.: US 2023/0031965 A1
(43) Pub. Date: Feb. 2, 2023

(54) INTRASACCULAR STENT DEVICE FOR ANEURYSM TREATMENT

(71) Applicant: Thushan Sivapatham, Litchfield, OH (US)

(72) Inventor: Thushan Sivapatham, Litchfield, OH (US)

(21) Appl. No.: 17/840,491

(22) Filed: Jun. 14, 2022

Related U.S. Application Data
(60) Provisional application No. 63/219,095, filed on Jul. 7, 2021.

(51) Int. Cl. A61B 17/12; A61F 2/958

(52) U.S. Cl. CPC A61B 17/12118; A61B 17/1214; A61F 2/958; A61B 2017/1205; A61B 2017/00871

(57) ABSTRACT

A process and system for treating an aneurysm using one or more specifically shaped endovascular coils and an intrasaccular stent/retaining device that operates to hold the one or more endovascular coils or other embolization devices within the aneurysmal sac of the aneurysm, thereby obviating the patients' need for lifetime dual anti-platelet therapy.

### CROSS-REFERENCE TO RELATED APPLICATIONS

This application claims the full Paris Convention benefit of, and priority to, United States provisional patent application serial numbered 63/219,095, and filed on Jul. 7, 2021.

### EXPRESSLY INCORPORATED BY REFERENCE

The following United States Letters Patents, PCT applications and all other attached publications are expressly incorporated by reference, as if fully set forth herein, to better define the state of neurovascular patent art, without any admission or preclusive status vis-à-vis the instant disclosures, they document the evolution of the devices and procedures comprising the Field as further developed below, disclosed, claimed and shown in the Appendices of the various documents incorporated by reference: PCT/US2014107090; PCT/US2015/014738; PCT/US2014/033881; and PCT/US2014/033881; U.S. Pat. Nos. 8,945,712; 8,926,680; 8,585,713; 8,945,143; 8,574,262; 8,545,415; 8,066,757; 8,070,791; 8,197,493; 8,088,140; 9,198,670; 9,186,151; PCT. US2018/019330; 7,757,582; 6,489,265; 6,953,672; 6,221,086; and, 6,083,220.

### BACKGROUND OF THE INVENTION

The subject invention is directed to a process and system for treating an aneurysm and more particularly, to a process and system for treating an aneurysm using one or more specifically shaped endovascular coils and a retaining device/intrasaccular stent (used interchangeably herein) that operates to hold the one or more endovascular coils within the aneurysmal sac of the aneurysm. An aneurysm is a weakened area in the wall of an artery and if it ruptures, it can result in life-threatening bleeding, and depending on its location, can cause damage to various parts of the body including brain damage. It is known that by preventing blood flow into the aneurysmal sac of an aneurysm, can significantly reduce the likelihood of the aneurysm rupturing. Accordingly, various devices and processes have been developed to reduce or prevent blood flow into the aneurysmal sac. One such method of reducing or preventing blood flow into the aneurysmal sac is the use of endovascular coiling (endovascular embolization). The process includes inserting a micro catheter into a groin artery of a patient and using a guidewire to direct the catheter to site of the aneurysm. Once the distal end of the catheter reaches the proper location at the aneurysm, the guidewire is removed by sliding it back through the catheter and one or more specifically shaped endovascular coils are introduced through the catheter, while in their stretched linear configuration, and are directed through the catheter towards its distal end. Using a pusher device, the endovascular coils are pushed out through the distal end of the catheter and are inserted into the aneurysmal sac by the pusher device exerting a force on the proximal end of the endovascular coils. Once inserted into the aneurysmal sac, the one or more endovascular coils are separated from the pusher device and assume their relaxed configuration thereby filling the aneurysmal sac to restrict blood flow into the aneurysmal sac thus reducing the likelihood of aneurysm rupture.

One problem that currently exists with using endovascular coiling for reducing or preventing blood flow into an aneurysmal sac is that a coil can migrate outwardly from the aneurysmal sac and into the parent blood vessel. This is particularly true with an aneurysm having a “wide-neck.” Such dislodging of a portion of an endovascular coil into the parent blood vessel can result in an occlusion creating a reduction of blood flow to areas of the patient's body and can also increase the likelihood of a blood clot forming in the parent blood vessel. Another problem often encountered with endovascular coils is that they often lack strength to maintain their relaxed configuration which can result in the endovascular coils not maintaining their desired position within the aneurysmal sac to prevent or significantly reduce blood flow into the aneurysmal sac thereby increasing the possibility of rupture or may result in causing the endovascular coils to move further out of the aneurysm.

Various types of systems that have been developed for maintaining endovascular coiling within an aneurysmal sac. One type of system uses a device that is placed within the lumen of the parent blood vessel such that it forms a wall along the outer mouth of the aneurysm to prevent the endovascular coiling from migrating through the neck of the aneurysm and into the parent blood vessel. One problem associated with such systems is that the stent device impedes blood flow through the parent blood vessel which can lead to blood clotting or reduction of blood flow through the parent blood vessel which can reduce oxygen levels in other areas of the patient.

Another system that has been developed for maintaining endovascular coiling within an aneurysmal sac and includes using a polymeric composition along the surface of the endovascular coiling such that the endovascular coiling when it expands into its relaxed configuration sticks to itself forming a rigid bundle which is too large to pass through the neck of the aneurysm. One problem associated with such a system is that it prevents easy adjustment or re-positioning of the endovascular coils once they expand into their relaxed configuration within the aneurysmal sac.

One system that has also been utilized to maintain endovascular coiling within an aneurysmal sac is the use of a mesh that is placed within the aneurysm sac to cover the inner mouth of the aneurysm. The mesh is positioned after the endovascular coiling has been inserted into the aneurysm sac. One problem associated with the use of such a mesh is that if the mouth of an aneurysm increases in size, the mesh itself could dislodge and travel through the neck of the aneurysm and enter the parent blood vessel or if dislodged, can allow a portion of an endovascular coil to escape into the parent blood vessel. In addition, it is often difficult to insert and properly position the mesh along the inner mouth of the aneurysm once the endovascular coil has been inserted into the aneurysmal sac.

Accordingly, it is desirable to have a process and system for treating an aneurysm that operates to prevent or reduce blood flow from entering the aneurysmal sac; that has a maintaining device that can be introduced into the aneurysmal sac using a micro catheter that is flexible and is sized to minimize any blockage of blood flowing through the parent blood vessel; that once deployed, the device or any portion of the device does not extend into the parent blood vessel; that operates to maintain endovascular coiling within the aneurysm sac; that cannot be or reduced the possibility of being dislodged or moved such that a portion of the endovascular coiling can escape and enter into the parent blood vessel; which can be easily adjustable; and will not be or reduces the possibility of being dislodged if the mouth and neck of the aneurysm increases in size.

The subject invention is a process and system for treating an aneurysm having an outer mouth, an inner mouth, a throat extending between the outer mouth and the inner mouth, and an aneurysmal sac. In an embodiment of the invention, the system and process include a catheter having an open distal end and a proximal open end and a removable guidewire extending through the catheter for directing the catheter into position such that the distal end of the catheter is located at the outer mouth of the aneurysm. A delivery wire having a distal end is sized to be inserted into the proximal open end of the catheter and can be moved towards the distal open end of the catheter. Removably attached to the distal end of the delivery wire is a retaining device such that when the distal end of the delivery wire is inserted through the neck and the inner mouth of the aneurysm, the retaining device expands outwardly into its relaxed configuration forming an expanded netting having a base and one or more fingers that expand longitudinally inwardly from the base into the aneurysmal sac. Once the retaining device has been properly positioned within the aneurysmal sac, the retaining device is separated from the delivery wire. Endovascular coiling is then directed into the aneurysmal sac through the expanded netting where the endovascular coiling expands from a compressed stretched configuration into a relaxed configuration and interweaves through the one or more fingers forming a cohesive structure.

The present inventions relate to the field of minimally invasive neurovascular interventions. In particular, the present inventions relate to endovascular delivery of devices to treat aneurysms, in complement to endovascular coils; whereby treatments and therapies deliver multiple potential devices to counteract the possibility of aneurysm rupture, which leads to hemorrhagic and ischemic stroke pathologies. Likewise, these inventions may be used secondary to any neurovascular interventions, such as in U.S. Pat. Nos. 9,808,359, and 10,814,045. In another embodiment of the invention, the system and process include a catheter having an open distal end and a proximal open end and a removable guidewire extending through the catheter for directing the catheter into position such that the distal end of the catheter is positioned within the aneurysmal sac. Once positioned, the guidewire is removed outwardly through the proximal open end of the catheter. A delivery wire is then inserted into the proximal end of the catheter and removably attached to the distal end of the delivery wire is a retaining device in its stretched position. Once the distal end of the catheter is in its desired position within the aneurysmal sac, the delivery wire operates to push the retaining device into the aneurysm sac. Thereafter, the retaining device expands into its relaxed configuration forming expanded netting having one or more fingers that extend into the aneurysm sac. The delivery wire can then be used to adjust the position of the netting and the one or more fingers such that once it is determined that the netting and the one or more fingers are properly placed within the aneurysmal sac, the delivery wire is separated from the retaining device. The delivery wire is then removed from the catheter and another delivery wire having a distal end connected to a pusher and one or more endovascular coils in their stretched compressed configuration. A second delivery wire then operates to guide a pusher attached to one or more endovascular coils to push the one or more endovascular coils into the aneurysmal sac where the one or more endovascular coils expand from a compressed stretched configuration into a relaxed configuration and interweaves through the one or more fingers forming a cohesive structure. Once the one or more endovascular coils and the one or more fingers have been interweaved together, the delivery wire then can then be used to make any additional desired adjustments to the position of the interweaved endovascular coils and the retaining device after which the pusher is separated from the endovascular coils and the catheter and delivery wire are removed from the patient.

In an embodiment of the invention, the system operates such that the retaining device is positioned within the aneurysmal sac after the endovascular coiling has been inserted into the aneurysmal sac.

In an embodiment of the invention, the one or more fingers each have a first end and a second end attached to the base forming a loop.

In an embodiment of the invention, the retaining device includes markers that are positioned along various endpoints of the expanded netting which operate to allow a physician to determine the position of the net within the aneurysmal sac.

In an embodiment of the invention, the distal ends of the ribs of the delivery device includes position markers for use in positioning the retaining device within the aneurysmal sac.

In an embodiment of the invention, the retaining device includes a base having a plurality of ribs extending outwardly from the base and netting webs secured to the plurality of ribs forming an umbrella shaped netting, and one or more fingers extend outwardly from the base.

In an embodiment of the invention the plurality of ribs and the one or more fingers are formed from a shape memory alloy.

In an embodiment of the invention, the retaining device forms an expanded umbrella shaped netting formed from a biocompatible shape memory polymer.

In an embodiment of the invention, the retaining device forms an expanded umbrella shaped netting formed from a polyurethane based polymer.

In an embodiment of the invention, the size of the retaining device is based on the size of the aneurysmal sac.

In an embodiment of the invention the netting includes at least one open space sized to allow the endovascular coiling to protrude in their compressed configuration through the at least one open space.

An embodiment the system for treating an aneurysm in a blood vessel, the aneurysm having an outer mouth, an inner mouth, a throat extending between the outer mouth and the inner mouth, and an aneurysmal sac, the system comprises: a catheter having an open distal end, a proximal open end and a lumen between the open distal and the proximal open end; a guidewire having a distal end and extends through the lumen of the catheter and operates to move through the blood vessel such that the distal end is positioned within the aneurysmal sac; a delivery wire having a distal end; a retaining device removable attached to the distal end of the delivery wire; and endovascular coiling; wherein the guidewire operates such that the catheter slides along the guidewire and directs the open distal end of the catheter into position within the aneurysmal sac; wherein the delivery wire operates to move through the lumen of the catheter and is removably attached to the retaining device such that when the distal end of the delivery wire is positioned within the aneurysmal sac, the retaining device expands outwardly into a relaxed configuration forming an expanded netting having a base and one or more fingers that expand longitudinally inwardly from the base into the aneurysmal sac; and wherein the endovascular coiling is directed into the aneurysmal sac through the expanded netting where the endovascular coiling expands into a relaxed configuration and interweaves through the one or more fingers forming a cohesive structure.

According to embodiments of the inventions, there are provided one or more fingers, each having a first end and a second end attached to the base forming a loop that operate to interweave with the endovascular coiling.

According to embodiments of the inventions, retaining devices/intrasaccular stents include markers positioned along various endpoints of the expanded netting, and operate to identify the position of the expanded netting within the aneurysmal sac.

According to embodiments of the inventions, the delivery device includes a base having a plurality of ribs extending outwardly from the base and netting webs secured to the plurality of ribs forming an umbrella shaped netting.

According to embodiments of the inventions, the plurality of ribs and the one or more fingers are formed from shape memory alloy.

These and other benefits, advantages, and embodiments of the invention will be apparent from the following description, the accompanying drawings and the appended claims, respectfully offered for consideration as new, novel and non-obvious, and eligible for United States Letters Patent, it is respectfully proposed.

### BRIEF DESCRIPTION OF THE FIGURES

Various features, aspects, and advantages of the present invention will become better understood with regard to the following description, appended claims, and accompanying drawings where:

FIG. 1 is a schematic illustration showing a parent blood vessel having an aneurysm extending from the parent blood vessel, the aneurysm includes an opening having an outer mouth, an inner mouth and a throat extending between the outer mouth and the inner mouth, and an aneurysmal sac extending outwardly from the inner mouth;

FIG. 2 is a schematic illustration of the system for treating an aneurysm showing a catheter having a guide wire slidably positioned within the lumen of the catheter, as illustrated the guide wire extends through the parent blood vessel and the opening of the aneurysm and into the aneurysmal sac for guiding the catheter through the opening of the aneurysm and into position within the aneurysmal sac;

FIG. 3 is a schematic illustration showing the open distal end of the catheter of the subject invention positioned at or near the inner mouth of an aneurysm, the guide wire having been removed;

FIG. 4 is a schematic illustration showing the proximal end of the catheter having a catheter inlet apparatus for inserting apparatus into the lumen of the catheter;

FIG. 5 is a schematic illustration showing the catheter of the subject invention having a retaining device in a compressed configuration within the lumen of the catheter and connected to the distal end of a delivery wire;

FIG. 6 is a schematic illustration showing the catheter of the subject invention showing the retaining device being pushed out through the open distal end of the catheter by the delivery wire;

FIG. 7 is a schematic illustration showing an embodiment of the invention of the retaining device formed from an elastic material expanded from its compressed configuration into its relaxed configuration forming a netting extending into the aneurysmal sac;

FIG. 8 is a schematic illustration showing the retaining device in its relaxed configuration having a base and a netting extending outwardly from the base and one or more fingers extending longitudinally outwardly from the base into the aneurysmal sac;

FIG. 9 is a schematic illustration showing an embodiment of one or more fingers extending outwardly from the base each finger having a first end connected

FIG. 10 is a schematic illustration showing another preferred embodiment of the retaining device having a plurality of netting webs with a plurality of spaces and a severable joint between the delivery wire for properly positioning the retaining device within the aneurysmal sac and the base of the retaining device;

FIG. 11 is a schematic illustration showing a second delivery wire within the lumen of a catheter attached to the proximal end of a pusher which is removably attached to the proximal end of an endovascular coil apparatus;

FIG. 12 is a schematic illustration showing the endovascular coiling being directed out through the open distal end of a catheter by a pusher and through a space in the netting web of the retaining device and the endovascular coiling beginning to expand into its relaxed configuration;

FIG. 13 is a schematic illustration showing the aneurysm with the endovascular coiling it is fully relaxed configuration filling the aneurysmal sac and the retaining device positioned over the inner mouth of the aneurysm; and

FIG. 14 is a schematic illustration showing the endovascular coiling being intertwined and entangled with the one or more fingers to form a cohesive structure.

### DETAILED DESCRIPTION OF THE INVENTION

The present inventor has crafted improvements regarding therapies complementary to, and in conjunction with, traditional endovascular coil delivery. Among the instant improvements are no materials breaching the aneurysmal sac, whereby all endovascular coils remain away from the parent blood vessel walls, precluding tissue insult, injury and damages. Accordingly, patients may be spared lifetime anti-clotting therapy intake.

### Exclusionary Embodiments

Excluded from the scope of the claimed subject matters are devices such as the prior art Web® devices (such as in U.S. Pat. No. 9,198,670) and Medina® systems, whose clinical performance has fallen short of keeping coils and embolization adjuncts, specifically to keep those bodies from coming out of aneurysms and collapsing therein.

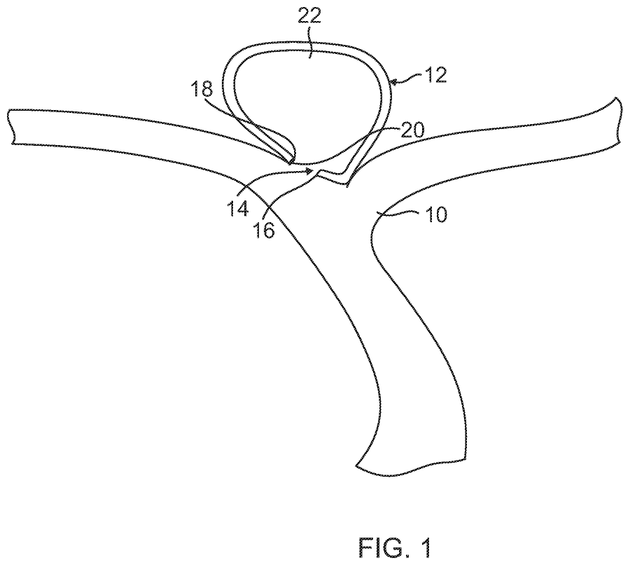

The subject invention is a process and system for treating an aneurysm such that endovascular coils can be placed within the aneurysmal sac and a retaining device operates to maintain the endovascular coils within the aneurysmal sac such that the retaining device does not extend into the parent blood vessel. Referring to FIG. 1 a parent blood vessel 10 is shown having an aneurysm 12 extending from the parent blood vessel 10. The aneurysm 12 includes an opening 14 having an outer mouth 16, an inner mouth 18, a throat 20 extending between the outer mouth 16 and the inner mouth 18, and an aneurysmal sac 22 extending outwardly from the inner mouth 18.

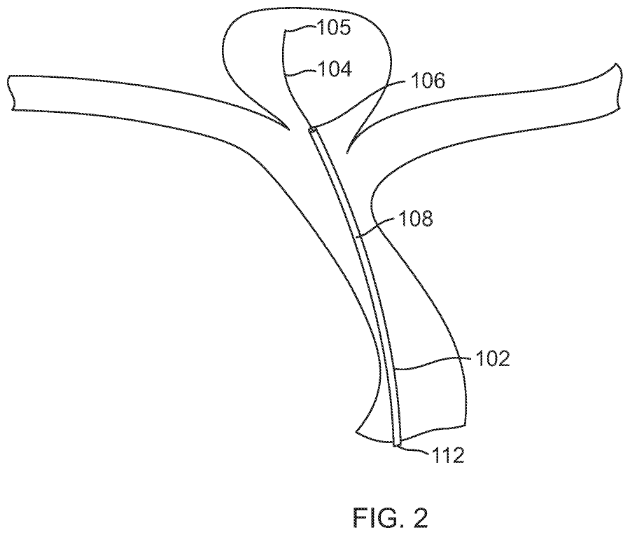

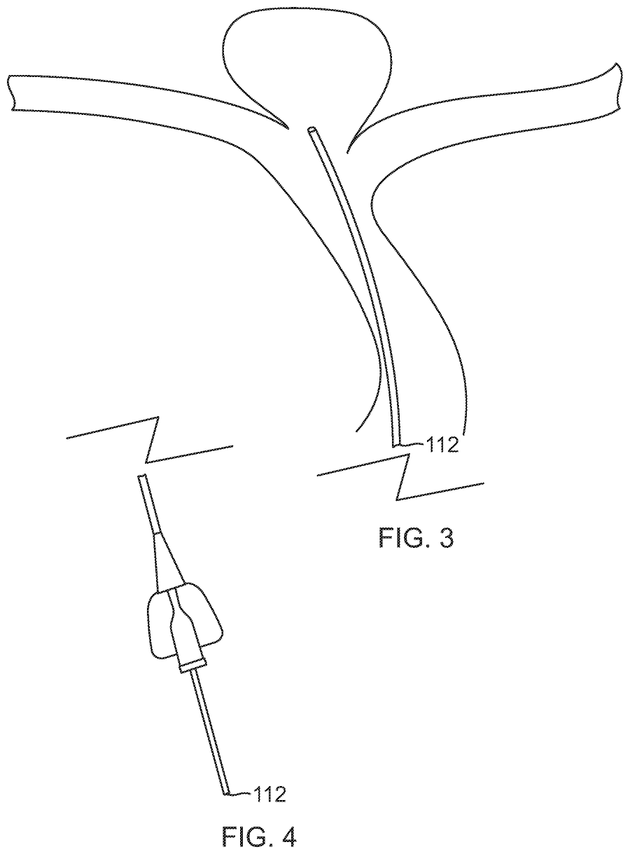

The system of the subject invention for treating an aneurysm is shown in FIG. 2 and includes a catheter 102 (micro catheter) having a guide wire 104 having a distal end 105 slidably positioned within the lumen 108 of the catheter 102. In operation, the guide wire 104 is inserted into an artery, generally near the groin of a patient, and is directed through the patient's vascular system towards the aneurysm 12. As illustrated, once the distal end 106 of the guide wire 104 is properly positioned near the opening 14 (or fully within the aneurysmal sac 22) of the aneurysm 12 inside the aneurysmal sac 22, the catheter 102 is then telescopingly slid over or moves along the guide wire 104 which directs the catheter 102 through the vascular system until the distal end 106 of the catheter 102 is placed near the opening 14 of the aneurysm 12 such that the distal open end 110 of the catheter 102 extends through the opening 14 of the aneurysm 12 and into the aneurysmal sac 22. Once the distal end 106 of the catheter 102 is in its proper position, the guide wire 105 can then be removed by withdrawing the guide wire 105 rearwardly out through the proximal open end 112 of the catheter 102 (FIG. 3). It should be understood that the catheter 102 of the subject invention is a conventional micro catheter typically used for treating aneurysms and may include a radiopaque material, such as blends of polymers and radiopaque additives, for example barium, bismuth and tungsten, that can be used to track the position of the catheter as it travels through a blood vessel of a patient.

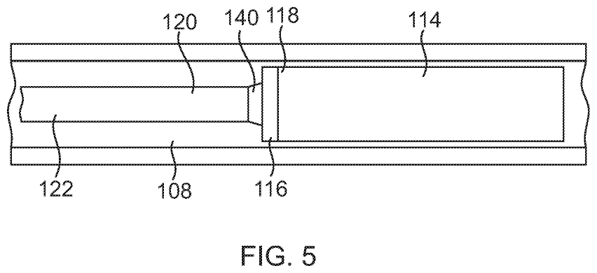

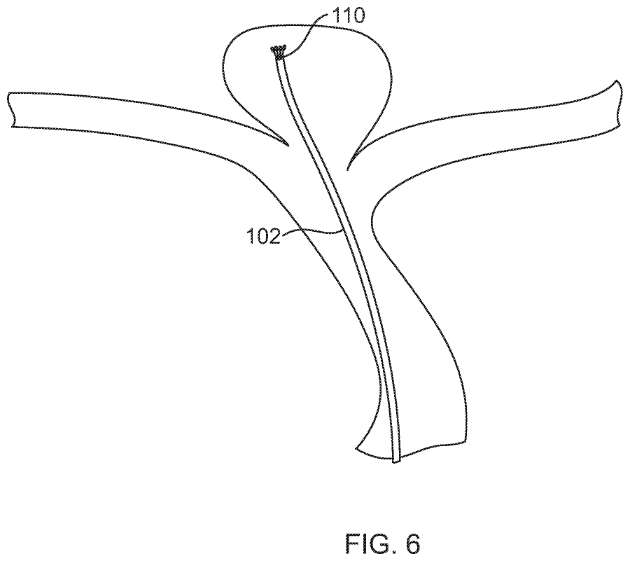

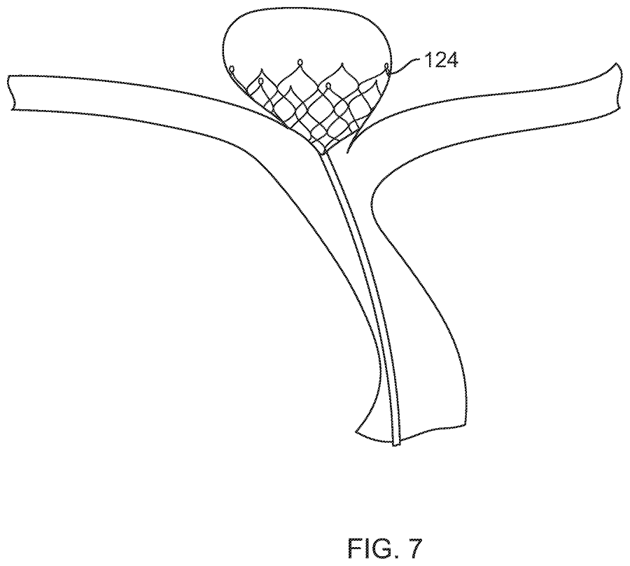

As illustrated in FIGS. 5-9, in an embodiment of the invention, after the distal open end 110 of the catheter 102 has been positioned in its desired location within the aneurysmal sac 22, a retaining device 114 is inserted into lumen 108 of the catheter 102 through its proximate open end 112. The retaining device 114 includes a base 116 at its proximal end 118 which is removable attached to the distal end 120 of a delivery wire 122. The retaining device 114 and the delivery wire 122 can then be pushed through the lumen 108 of the catheter 102 by the delivery wire 122 until it reaches the distal open end 112 of the catheter 102 (FIG. 6). As shown in FIG. 5, the retaining device 114 is formed such that it can be placed into a compressed stretched configuration so that it can be pushed by the delivery wire 122 through the lumen 108 of the catheter 102 until it reaches the distal open end 110 of the catheter 102 (FIG. 6) whereupon the delivery wire 122 will push the retaining device 114 out through the distal open end 110 of the catheter 102. Once the retaining device 114 is pushed out through the distal open end 110 of the catheter 102 it will expand outwardly from the base 116 into a relaxed configuration (FIG. 7) forming an expanded umbrella shaped netting 124 with one or more fingers 126 that expand longitudinally outwardly from the base 116 into the aneurysmal sac 22.

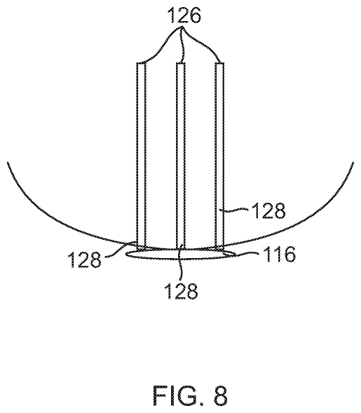

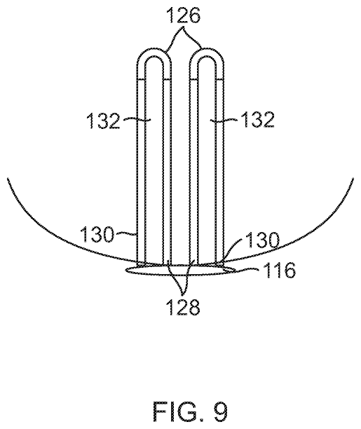

As illustrated in FIGS. 8 and 9, when the retaining device 114 has expanded into its relaxed configuration the expanded umbrella shaped netting 124 is disposed over and covers the inner mouth 18 of the aneurysm 12 (FIG. 7). In an embodiment, each of the one or more fingers 126 extending outwardly from the base 116 into the aneurysmal sac 22 includes a first end 128 attached to the base 116 (FIG. 8). In another preferred embodiment, as illustrated in FIG. 9, each of the one or more fingers 126 extending outwardly from the base 116 into the aneurysmal sac 22 includes a first end 128 attached to the base 116 and a second end 130 attached to the base 116 such that each finger 126 forms an open loop 132 (FIG. 9).

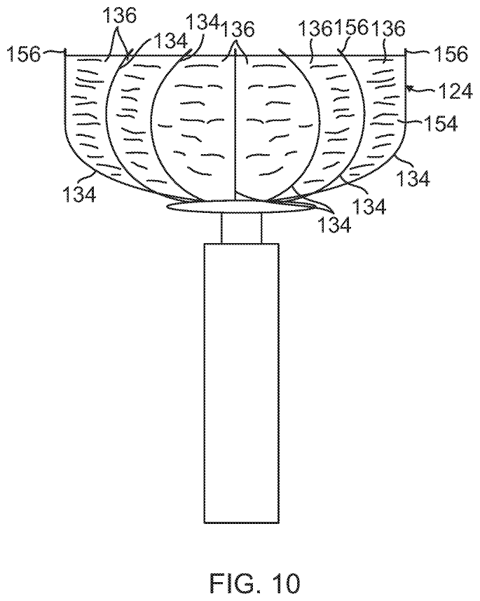

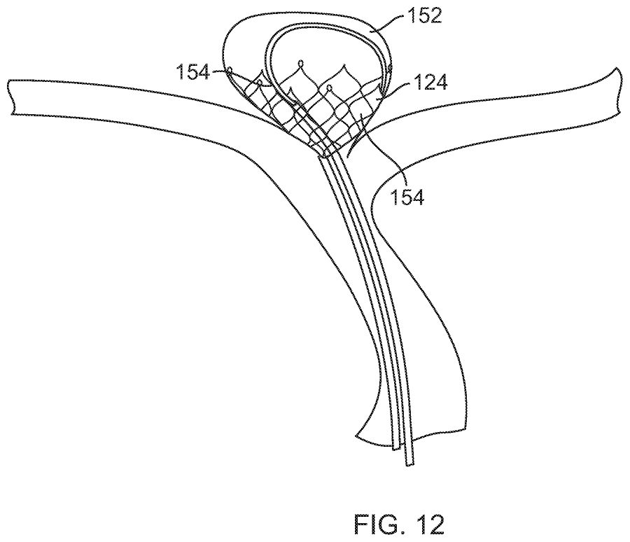

As illustrated, in an embodiment, as illustrated in FIG. 10, the umbrella shaped netting 124 includes a plurality of ribs 134 extending radially outwardly from the base 116 for supporting a plurality of netting webs (panels) 136. Preferably, the netting webs 136, the one or more fingers 126 and the ribs 134 are formed from materials that can be placed into compressed stretched configuration (FIG. 5) and can slide through the lumen 108 of the catheter 102 and will cooperate together to expand into a preprogrammed relaxed configuration when expelled from the distal open end 110 of the catheter 102. In an embodiment, the one or more fingers 126 and the plurality of ribs 134 are formed from a super-elastic material. Such super-elastic materials are known as shape memory alloys, such as Nitinol (NiTi), that have the ability to go from an austenitic crystal structure to a stress induced martensitic structure at a specified temperature and then transform elastically back to its austenitic shape when stress is removed. It should be understood to one skilled in the art that various super-elastic materials have been developed that maybe utilized and include nickel/titanium (Nitinol) alloys, nickel/aluminum alloys, and copper/zinc alloys. In another preferred embodiment, the umbrella shaped netting 124 is formed from a biocompatible, non-toxic elastic memory polymer or functional material, as illustrated in FIGS. 7 and 12, such as a polyurethane based polymer that can be compressed for insertion through a catheter and will return to its relaxed configuration when it is inserted into the aneurysmal sac and heated above its transition temperature thereby expanding into an umbrella shaped netting.

In an embodiment, as illustrated in FIG. 10, the netting webs 136 are attached to the radially outwardly extending ribs 134 and are an open spaced weave having open spaces 135 formed from a vascular graft material, such as polyethylene terephthalate (“Dacron”), or polytetrafluoroethylene (“Teflon”) or polyurocarbons. As stated, the retaining device 114 is designed such that the fingers 126, ribs 134 and the netting webs 136 can be placed in a substantially linear compressed configuration (FIG. 5) when placed within the lumen 108 of the catheter 120 and are sized so they can be moved through the lumen 108 of the catheter 120 and placed in position in the aneurysmal sac 22 where the fingers 126 and the ribs 134 assume their relaxed configuration forming the umbrella shaped netting 124 (FIGS. 7 and 8). In an embodiment, the fingers 126 and the ribs 134 are placed into the catheter at a temperature below the transition temperature of the shape-memory alloy(s) forming the ribs 134 and fingers 126 such that they are flexible and can be stretched into a linear configuration so that they can be moved through the lumen 108 of the catheter 120 and deployed in the aneurysmal sac 22 where they will be heated above their transition temperature causing the ribs 134 and fingers 126 to expand into their relaxed configuration forming the umbrella shaped netting 124 with one or more fingers 126 extending into the aneurysmal sac 22.

After the retaining device 114 has been properly positioned and deployed within the aneurysmal sac 22, the delivery wire 122 is detached from the base 116 of the retaining device 114. Various methods have been developed, both mechanical and electrical, for detaching the delivery wire from the base. As shown in FIG. 5, preferably, the distal end 120 of the delivery wire 122 includes an electrolytically severable joint 140 such that when the retaining device 114 is properly positioned within the aneurysmal sac 22, a small voltage is provided through the delivery wire 122 causing the joint 140 to erode thereby electrolytically detaching the delivery wire 122 from the base 116 of the retaining device 114.

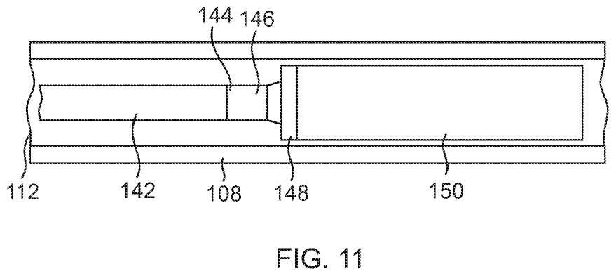

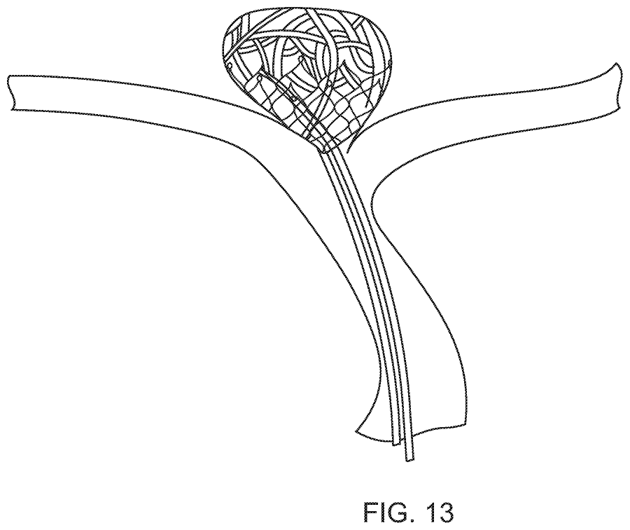

After the retaining device 114 has been positioned and deployed within the aneurysmal sac 22 and the delivery wire 122 has been detached from the base 116, the delivery wire 122 can then be removed by withdrawing the delivery wire 122 rearwardly out through the proximal open end 112 of the catheter 102. In an embodiment, a second delivery wire 142 (FIG. 11) is inserted into lumen 108 of the catheter 102 (or a second catheter) through its proximate open end 112. The second delivery wire 142 is attached to the proximal end 144 of a pusher 146 which is removably attached to the proximal end 148 of an endovascular coil apparatus 150 (FIG. 11). The endovascular coil apparatus 150 comprises one or more endovascular coils 152 (FIG. 12) that are typically used for treating aneurysms and have a regular or helical configuration that operate to form a physical barrier to blood flowing into the aneurysmal sac. Preferably, the one or more endovascular coils 152 are made from a shape-memory alloy of a radiopaque, biocompatible metal, such as platinum, gold, silver, tungsten or an alloy of such materials combined with a shape-memory alloy and are designed such that they are compressed and substantially linear (FIG. 11) when placed within the lumen 108 of the catheter 102 and sized so they can be moved through the lumen 108 of the catheter 102 and placed in position in the aneurysmal sac 22 where they assume their relaxed configuration to fill the aneurysmal sac 22 (FIG. 13). In an embodiment, the endovascular coils 152 are placed into the catheter 102 at a temperature below the transition temperature of the shape-memory alloy forming the endovascular coils 152 such that the coils are flexible and can be stretched into a linear configuration so that they can be moved through the lumen 108 of the catheter 102 and deployed in the aneurysmal sac 22 where the shape-memory alloy will be heated above its transition temperature causing the endovascular coils 152 to expand into their relaxed configuration filling the aneurysmal sac 22.

In another preferred embodiment of the invention, as shown in FIG. 13, the endovascular coils and the retaining device and be deployed into the aneurysmal sac using separate catheters, one for deploying the endovascular coils and one for deploying the retaining device. It should also now be understood that the use of two separate catheters permits the deployment and the positioning of the endovascular coils and the retaining device to be more easily positioned relatively to each other and allow for any necessary adjustments to be more easily made.

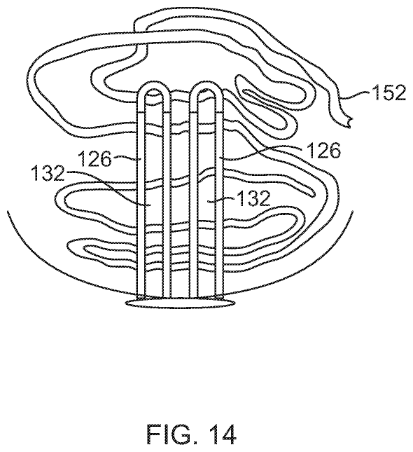

After the retaining device 114 has been properly placed within the aneurysmal sac 22, the endovascular coils 152 can be directed through a space (opening) 154 in a netting web 136 (FIGS. 10 and 12) and positioned near the one or more fingers 126 of the retaining device 114 such that when the endovascular coils 152 expand into their relaxed configuration (FIG. 13), they will intertwine and entangle themselves with the one or more fingers 126 while filling the aneurysmal sac 22 (FIG. 14). For a non-limiting example, the endovascular coils 152 can be positioned such that when they expand into their relaxed configuration that move through one or more loops 132 formed by the one or more fingers 126. This expansion and intertwining process results in the endovascular coils and the retaining device forming a cohesive structure which is too large to pass through the opening 14 of the aneurysm. Further, the umbrella shaped netting of the retaining device positioned across the inner mouth 18 of the aneurysm 12 prevents any part of the endovascular coils from passing through the opening and extending into the parent blood vessel. It should also be understood that the intertwining and entangling of the endovascular coils with the retaining device helps maintain the retaining device across the inner opining of the aneurysm and the endovascular coils in position within the aneurysmal sac.

In another preferred embodiment of the invention, as illustrated in FIG. 10, the ribs 134 each include a position marker 156 at the distal end 158 of each rib 134. In another preferred embodiment, netting formed by a memory shaped polymer, such as shown in FIG. 12, includes position markers 156 that can be placed at various locations along the netting. The position markers 156 operate such that during the positioning of the retaining device 114, the markers 156 allow the physician to determine if the retaining device is properly positioned within the aneurysmal sac. The markers can be formed from various biocompatible, radiopaque materials and can be sized or the material selected that makes the markers distinguishable over the material forming the ribs.

It should now be understood that the process and system for treating an aneurysm of the subject invention operates to prevent or reduce blood flow from entering the aneurysmal sac; that provides a system and process for reducing blood flow entering into the aneurysmal sac while minimizes or eliminates obstruction of the parent blood vessel; that includes a retaining device that cooperates with an endovascular coil apparatus to reduce or eliminates blood flow entering into the aneurysm sac. It should now be apparent to one skilled in the art that the system of the subject invention allows a retaining device to be properly positioned across the inner mouth of an aneurysm while allowing an endovascular coil apparatus to be inserted into the aneurysm sac. Further, the retaining device and endovascular coils cooperate together to form a cohesive structure that operates to reduce blood flow into the aneurysm sac while reducing or eliminating the likelihood that an endovascular coil or a portion thereof will migrate out through the opening in the aneurysm and into the parent blood vessel. In addition, the system of the subject invention allows for the physician to monitor the location of the retaining device to ensure that the retaining device is in the proper position and allows the position of the retaining device and the endovascular coils to be adjusted after they are placed in the aneurysmal sac.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary, and that the actual parameters, dimensions, materials, and/or configurations will depend upon the specific application or applications for which the teachings of the present disclosure is/are used.

Those skilled in the art will recognize or be able to understand that wires of the instant systems, like the prior art disclosed in the “Expressly Incorporated by Reference” section maybe be straight, and/or pre-shaped (for example, 45 degrees, 90 degrees, or J-shaped) and span all wire sizes approved for endovascular use. Likewise, it is readily understood that electrolytic, thermal, or mechanical detachment, along with later developed technologies, is covered. Similarly, catheters maybe include balloon tips for stability and placement. Finally, steps are interchangeable. For example, artisans understand that the retaining member/intrasaccular stent may be detached after coils are placed with a second catheter—meaning the present invention is detachable before, during, and after placements of adjunctive therapies, including endovascular coils.

Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment,” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention, and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O interface may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antennae, and a network interface device (e.g., a network interface card (NIC), Wi-Fi card, cellular modem, data jack, Ethernet port, modem Jack, HDMI port, mini-HDMI port, USB port), touchscreen (e.g., CRT, LCD, LED, AMOLED, Super AMOLED), pointing device, trackpad light (e.g., LED), light/image projection device, or a combination thereof.

Memory according to the invention refers to a non-transitory memory, which is provided by one or more tangible devices which preferably include one or more machine readable medium on which is stored one or more sets of instructions (e.g., software) embodying any one or more of the methodologies or functions described herein. The software may also reside, completely or at least partially, within the main memory processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the it1 vem ion includes a tangible, non-transitory computer readable medium for memory. Exemplary devices for use as memory include semiconductor memory devices, (e.g., EPROM, EEPROM, solid state drive (SSD), and flash memory devices, (e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A system for addressing neurovascular issues, which comprises, in combination:
at least an intrasaccular stent/retaining device;
reliably emplaceable and stably fixed within an aneurysm; and
complementary therapies used in conjunction therewith;
further comprising at least an endovascular coil, and other embolization devices.

2. The system of claim 1, wherein the at least an intrasaccular stent/retaining device maintains all emplaced medical device therapies within the confines defined as the aneurysmal sac, once delivered.

3. The system of claim 2, whereby once emplaced, the intrasaccular stent/retaining device maintains all emplaced medical device therapies within the confines defined as the aneurysmal sac, thereby reducing the need for dual anti-platelet therapy for the rest of the patient's life.

4. The system of claims 1-3, the intrasaccular stent/retaining device further comprising a base effective to contain all delivered coils inside of the neck of the subject aneurysm without protrusion, including balloon-tipped catheter to support compliance with angulation of the neck of an aneurysm.

5. The system of claims 1-4, consisting essentially of one metal and markers selected from the group of platinum, stainless steel, cobalt-chrome alloy, titanium, and nickel-titanium alloy (nitinol). Other metals, such as gold, silver, iridium, tantalum, and tungsten, maybe used, as well as those comprised of shape memory alloys (NiTi™—Nitinol®).

6. The system of claims 1-5, further comprising netting composed of biocompatible, non-toxic shape memory polymers.

7. The system of claims 1-5, further comprising expanded netting formed from polyurethane.

8. The system of claims 1-6, whereby all endovascular delivery and all intrasaccular stent/retaining device member deployment resides within the aneurysm, whereby no elastic or plastic deformation permits collapse of a resulting structure.

9. The system of claim 8, whereby obstruction of a parent vessel is eliminated, all coils and intrasaccular stent/retaining device elements being lodged within the aneurysmal sac, including 45 degrees, 90 degrees, or J-shaped, as approved for endovascular surgery, with electrolytic, thermal, mechanical, or other type of coil detachment; likewise no order of delivery or emplacement is required; including, intrasaccular stent/retaining device being detachable before, during, or after placement of adjunctive therapies, coils, and other devices.

10. The system of claim 9, whereby, irrespective of the aneurysmal neck-type, the inner mouth of the aneurysm defines an inviolate border from the parent vessel, as confirmed by fluoroscopy.

11. The system of claim 10, whereby a complex is formed by the intrasaccular stent/retaining device, being a cohesive structure operating to reduce blood flow into the aneurysmal sac, wherein optionally there is a covered stent component on a proximal portion of the device.

12. The system of claim 11, further functioning to reduce or eliminate risks of anything migrating out of the former opening of the aneurysm.

13. The system of claim 12, proper positioning being confirmable by evidence of imaging, and comprising a dataset including completion angiography.

14. The system of claim 13, the dataset showing fully relaxed endovascular coiling and filled aneurysmal sac, with intrasaccular stent/retaining device positioned over the inner-mouth of an aneurysm being treated, sorted by AI and mediated by humans, to a protected, patient-blind HIPAA compliant patient registry and private database.

15. An app and associated special-purpose computer which arrays the datasets of claim 14 such that it can be used to update sustaining engineering data, to optimize the system.

16. An interface to communicate datasets to any required healthcare provider at patients' request, using a Blockchain as needed.

17. A system for treating an aneurysm in a blood vessel, the aneurysm having an outer mouth, an inner mouth, a throat extending between the outer mouth and the inner mouth, and an aneurysmal sac, the system comprises:
a catheter having an open distal end, a proximal open end and a lumen between said open distal and said proximal open end;
a guidewire having a distal end and extends through said lumen of said catheter and operates to move through the blood vessel such that said distal end is positioned within the aneurysmal sac;
a delivery wire having a distal end;
a intrasaccular stent/retaining device removable attached to said distal end of said delivery wire; and
endovascular coiling;
wherein said guidewire operates such that said catheter slides along said guidewire and directs said open distal end of said catheter into position within said aneurysmal sac;
wherein said delivery wire operates to move through said lumen of said catheter and is removably attached to said intrasaccular stent/retaining device such that when said distal end of said delivery wire is positioned within the aneurysmal sac, said intrasaccular stent/retaining device expands outwardly into a relaxed configuration forming an expanded netting having a base and one or more fingers that expand longitudinally inwardly from said base into the aneurysmal sac; and
wherein said endovascular coiling is directed into the aneurysmal sac through said expanded netting where said endovascular coiling expands into a relaxed configuration and interweaves through said one or more fingers forming a cohesive structure.

18. The system of claim 17, wherein said one or more fingers each have a first end and a second end attached to the base forming a loop.

19. The system of claim 17, wherein said retaining device includes markers positioned along various endpoints of said expanded netting and operate to identify the position of said expanded netting within the aneurysmal sac.

20. The system of claim 17, wherein said retaining device is sized based on the size of the aneurysmal sac.
